(19) United States
(12) Patent Application Publication
(10) Pub. No.: US 2015/0128865 A1
(43) Pub. Date: May 14, 2015

(54) Entomopathogenic nematode do-it-yourself application and rearing system

(71) Applicant: Allison Hope Justice, Fair Play, SC (US)

(72) Inventor: Allison Hope Justice, Fair Play, SC (US)

(21) Appl. No.: 14/080,516

(22) Filed: Nov. 14, 2013

(51) Int. Cl. A01K 67/033; A01K 29/00

(52) U.S. Cl. CPC A01K 67/033; A01K 29/00
USPC 119/6.7

(57) ABSTRACT

A composition and method for biological control of common greenhouse, nursery, and mushroom house pests using entomopathogenic nematodes. The method contains kits for rearing nematodes at the end user facility. Unlike the current temperature limited nematode applications only using one species, multiple genera and species of nematodes will be used.

### FIELD OF THE INVENTION

The present invention relates to the biological control “do-it-yourself” application and rearing system of a mixture of Entomopathogenic nematode species to control various destructive horticultural insect pests.

### BACKGROUND OF THE INVENTION

Currently, the desire to use less chemical pesticides has increased while disease and pest pressure is static. Costs for pest control compromises a large portion of farming production costs. The switch to biological control, or method of using living organisms to control other living organisms, is increasing steadily. The price to use such living organisms is generally higher than chemical control due to price to produce live agents and to keep them viable during shipment. Live biological control shipment usually occurs through overnight shipment wherein the agent is kept in a cool dormant state. Various biological control agents are currently used in the field of agriculture such as nematodes, fungi, bacteria, virus, and insects.

Entomopathogenic nematodes, or microscopic round worms, are one current method used by plant and mushroom producers to control fly larvae that can disrupt plant root or mycelial systems. Nematodes in the genera Steinernema and Heterorhabditis are obligate parasites of many insect juvenile stages that affect plant roots or mycelium. These nematodes are currently commercially available by companies in a carrier (gel, water, sponge) and then made into an aqueous suspension and applied with a sprayer. (Caamano, Quality Assessment of Two Commercially Available Species of Entomopathogenic Nematodes: Steinernema feltiae and Heterorhabditis indica. HortTechnology. January-March 2008 18(1); Gouge, Glasshouse control of fungus gnats, Bradysia pauper, on fuchsias by Steinernema feltiae. Fundam. Appl. Nematol., 1995, 18(1), 77-80.) Nematodes enter insect hosts through natural openings in which after they release a symbiotic bacterium (Xenorhabdus spp. for Steinernema spp. and Photorhabdus spp. for Heterorhabditis spp.) causing host death within 48 hours. Inside the host the nematodes complete 2-3 generations and the final stage of infective juveniles (IJs) emerge. Tens to hundreds of thousands of IJs can emerge depending on the insect host. These emergents are the only stage of nematodes that can survive and infect other insects in a natural environment. (Christen, Responses of the entomopathogenic nematode, Steinernema riobrave to its insect hosts, Galleria mellonella and Tenebrio molitor. Parasitology (2007), 134, 889-898; Kaspi, Foraging efficacy of the entomopathogenic nematode Steinernema riobrave in different soil types from California citrus groves. Applied Soil Ecology 45 (2010) 243-253.)

Nematodes can be produced though mass production through in vivo or in vitro methods. To reduce costs the majority of companies will grow nematodes without a living host in large fermenters. Once produced, the IJs are put into a dormant stage by cold-storage and shipped to the end user. By putting the IJs into cold storage, the later infectivity to a new host is decreased. (Boff, Effect of storage time and temperature on infectivity, reproduction and development of Heterorhabditis megidis in Galleria mellonella. Nematology, 2000, Vol. 2(6), 635-644.) Another way to rear nematodes is to infect a host and later collect the IJs that emerge. In vivo production has been thought to be more expensive than mass fermenter production but this is not accurate in the present invention.

In current greenhouse, nursery, and mushroom production the prevalent nematode used is Steinernema feltiae. This is due the commercial availability and also success with controlling Sciarid insects. The problem with only using S. feltiae is that it is a cold-adapted species and is only infective at temperatures around 10-25 C and can only reproduce at temperatures between 12-25 C. (Molynex, Heterorhabdistis spp. and Steinernema spp.: Temperature, and Aspects of Behavior and Infectivity. 1986, Experimental Parasitology 62, 169-180.) Greenhouses, nurseries, and mushroom houses commonly are grown at temperatures higher than 25 C, especially during the summer months. This constraint of only using one cold-adapted nematode restricts efficiency. Including other warm-adapted species of nematodes such as S. carpocapsae, H. bacteriophora, S. riobrave, H. indica and H. zealandica could increase the efficiency of insect control and reproduction rate at higher temperatures. (Jagdale, Effect of entomopathogenic nematode species, split application and potting medium on the control of the fungus gnat, Bradysia difformis (Diptera: Sciaridae), in the greenhouse at alternating cold and warm temperatures. 2007, Biological Control 43,23-30.)

The biggest constraints on growers in using nematodes are cost, temperature effecting efficacy, and reduced infectivity due to cold-storage. Though the science of nematode production has progressed there is a need for a more cost effective way to provide viable entomopathic nematodes for biological control.

### SUMMARY OF INVENTION

This disclosure is directed to a system for an end user to obtain a nematode breeding kit and produce virulent nematodes of different species on site. This kit includes a syringe and cups of insect host larvae that will be shipped to the user. A cocktail of nematode genera and species will be used in order to overcome the temperature fluctuations in a growing environment. The user will then inoculate their host larvae cups with the nematodes and give them 14 days to complete their cycle. At day 14 the user will use a sieve and water to separate the emerged IJs from the insect hosts and sawdust. This final filtered solution can then be applied to plants or mushrooms.

### BRIEF DESCRIPTION OF THE ACCOMPANYING FIGURES

FIG. 1. Is a flowchart which describes the components of the kit, procedures and timeline after the end user acquisition, and application of the final product.

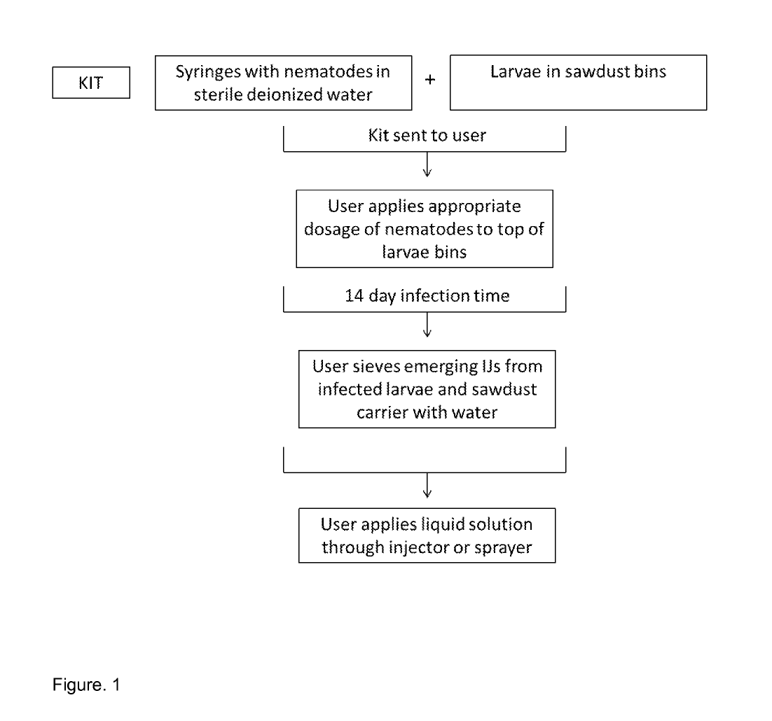

### DETAILED DESCRIPTION OF THE INVENTION

The present invention is for providing large numbers of different genera and species nematodes that are never placed in cold storage. This process is a replacement for mass production systems that produce nematodes in a fermenter. This process is cost effective in that the end user will be rearing the nematodes on site.

Various arthropods can be used as the insect host including larvae in the family Tenebrionidae, e.g., Tenebrio molitor, Zophobas mario, and Alphitobus diaperinus, as well as the maggot stage of Calliphora vomitoria and larval stage of Galleria mellonella. Host insect juveniles will be provided in an ordinary fishing worm bin in sawdust. The area the user wants to inoculate in the greenhouse, nursery, or mushroom house will determine the number of bins and nematodes sent in a kit. Each insect juvenile will receive 50 IJs for proper inoculation. The syringe will not contain a needle. It will be used simply as a way to measure and apply the correct dosage to the bins. Bins will be inoculated by applying correct amount of nematode solution on top of sawdust carrier.

For example, if a user wants to treat 550 ft² he would order 500 insect host juveniles and 10,000 IJs. This would yield 50 million IJs for the suggested 550 ft² treatment area.

The IJs in the syringe would be a mix of cold and warm-adapted nematodes such as S. carpocapsae, H. bacteriophora, S. riobrave, H. indica, S. feltiae, and H. zealandica. Each bin would be inoculated by only one species and later combined after sieving for easy application. It will require 14 days at room temperature for insect hosts to become inoculated, colonized, and emerge. Nematodes for syringes will reared the same way as mentioned above.

The primary targeted hosts for nurserymen, greenhouse and mushroom house growers are larvae of Sciardis (Lycoriella mali, Bradysia spp.), Cecid flies (Mycophila speyeri, Heteropeza pygmaea), Musca domestica, Megaselia halterata, Stomoxys calcitrans, Franklinothrips spp. and Scatella stagnalis.

What is claimed is:

1. A system for delivering biological entomopathic nematodes as a rearing kit to the end user.

2. The system of claim 1 wherein the kit compromises syringes of nematodes and worm bins of larvae.

3. The system of claim 2 wherein the syringes contains sterilized water and different genera and species of nematode.

4. The system of claim 2 wherein the individual syringes contain S. carpocapsae, H. bacteriophora, S. riobrave, H. indica, S. feltiae, and/or H. zealandica.

5. The system of claim 1 wherein the worm bin contains juvenile stages of Tenebrio molitor, Zophobas mario, Alphitobus diaperinus, Calliphora vomitoria, or Galleria mellonella in a saw dust carrier.

6. The system of claim 1 wherein the kit is transferred to the end user for rearing.

7. The method of claim 6 wherein the user inoculates the bins with nematodes at a rate of 50 IJ/host.

8. The method of claim 7 wherein the user places the bins in a dark place in room temperature for 14 days.

9. The method of claim 8 wherein the user sieves the saw dust carrier and infected hosts to separate the emerged IJs.

10. The method of claim 9 wherein the user applies this solution to targeted area.
